(19) United States
(12) Patent Application Publication
(10) Pub. No.: US 2023/0256272 A1
(43) Pub. Date: Aug. 17, 2023

(54) AIR PURIFICATION APPARATUS FOR NASAL APPLICATION AND ASSOCIATED CASE

(71) Applicant: James L. Orrington, II D.D.S., P.C., Chicago, IL (US)

(72) Inventors: James L. Orrington, II, Flossmoor, IL (US); Janie Orrington-Myers, Terre Haute, IN (US); Brandi Marie Henry, Crystal Lake, IL (US)

(21) Appl. No.: 17/994,293

(22) Filed: Nov. 26, 2022

Related U.S. Application Data
(63) Continuation-in-part of application No. 17/982,355, filed on Nov. 7, 2022.
(63) Continuation-in-part of application No. 29/849,707, filed on Aug. 12, 2022.
(60) Provisional application No. 63/310,650, filed on Feb. 16, 2022.

(51) Int. Cl. A62B 23/06; A61L 9/20; A61L 9/12; A61L 9/014; A61M 15/08; A62B 7/10; A62B 25/00; A62B 9/00

(52) U.S. Cl. CPC A62B 23/06; A61L 9/014; A61L 9/20; A61L 9/122; A61M 15/08; A62B 7/10; A62B 9/00; A62B 9/006; A62B 25/00; A61L 2209/12; A61L 2209/14; A61L 2209/22; A61L 2209/111; H02J 7/0042

(57) ABSTRACT

An air purification apparatus for nasal application is provided for purifying and/or reducing undesirable materials from air intended to enter a nasal cavity. The air purification apparatus for nasal application may include an air purification apparatus, housing component, power component, fan component, filtration component, germicidal irradiation component, aroma component, olfactory substance delivery component, nose plug component, monitoring component, and case. The case may reversibly enclose the apparatus and provide electrical power delivery.

### CROSS-REFERENCE TO RELATED APPLICATION

This application claims priority from U.S. provisional Pat. application serial number 63/310,650 filed Feb. 16, 2022. This application also claims priority from U.S. Pat. application serial number 29/849,707 filed Aug. 12, 2022. This application also claims priority from U.S. Pat. application serial number 17/982,355 filed Nov. 7, 2022. The foregoing applications are incorporated in their entirety herein by reference.

### FIELD OF THE INVENTION

The present disclosure relates to an air purification apparatus for nasal application. More particularly, the disclosure relates to purifying and/or reducing undesirable materials from air intended to enter a nasal cavity.

### BACKGROUND

For generations, and most particularly since the onset of the COVID-19 pandemic, societies have been cognizant of environmental conditions that may present health hazards. Today, vigilance in the face of air-borne viruses and other hazardous agents is prevalent throughout the world. Devices that help prevent inhalation of viruses and other harmful agents in the environment are in high demand by those seeking to maintain optimal health and not contract COVID-19 or other harmful airborne viruses and agents.

Carbon air filters are well-known to be effective in removing hazardous gaseous chemicals, as well as certain odors, from the air. HEPA (high efficiency particulate absorbing) air filters, by contrast, are mechanical in nature. HEPA filters are well-known to remove undesired particulate matter from the air. Those of skill in the art readily appreciate that carbon air filters and HEPA filters may be used together.

Ultraviolet (UV) light consists of electromagnetic radiation with wavelengths shorter than visible light but longer than x-rays. UV light is well-known to exhibit anti-viral and anti-bacterial properties. Short wavelength UV light (UV-C) is considered “germicidal UV.” Nucleic acids that make up DNA of viruses and bacteria may exhibit defects when exposed to UV-C light. These defects may prevent effective replication, thus eliminating or substantially reducing the ability of viruses and bacteria to replicate.

The sense of smell plays a significant role in the physiological effects of mood, stress, and working capacity. A large family of olfactory receptors present in humans are genetically designed to detect thousands of different fragrances. The positive impact of prolonged exposure to certain types of aromas and aromas on psychophysiological activities of humans has been recognized for years. See, e.g., K. Sowndhararajan & S. Kim, Influence of Fragrances on Human Psychophysiologylogical Activity With Special Reference to Human Electroencephalographic Response, 84(4) Sci. Pharm. 724 (2016). Therefore, a need exists to solve the deficiencies present in the prior art.

What is needed is an apparatus to remove contaminants, particulates, and other undesirable elements from air prior to inhalation by a user. What is needed is an air purification apparatus to reduce contact of undesirable elements with tissue within a nasal cavity or otherwise related to the respiratory system. What is needed is a device to remove impurities from air prior to entering the nasal cavity by performing filtration and/or germicidal irradiation. What is needed is a portable device to be comfortably inserted into a nostril and perform air purification functions. What is needed is a rechargeable air purification device that can detect a condition relating to incoming air and at least partially purify same.

### SUMMARY

An aspect of the disclosure advantageously provides an apparatus to remove contaminants, particulates, and other undesirable elements from air prior to inhalation by a user. An aspect of the disclosure advantageously provides an air purification apparatus to reduce contact of undesirable elements with tissue within a nasal cavity or otherwise related to the respiratory system. An aspect of the disclosure advantageously provides a device to remove impurities from air prior to entering the nasal cavity by performing filtration and/or germicidal irradiation. An aspect of the disclosure advantageously provides a portable device to be comfortably inserted into a nostril and perform air purification functions. An aspect of the disclosure advantageously provides a rechargeable air purification device that can detect a condition relating to incoming air and at least partially purify same.

Accordingly, the disclosure may feature an air purification apparatus for nasal application comprising a housing, air treatment components, power component, and a case. The housing may substantially enclose an interior space. The air treatment components may be located within the interior space to at least partially purify air for delivery to a nasal cavity. The air treatment components may include a filtration component to remove impurities from the air and a fan component to actively pass the air through the filtration component. The power component may be located substantially within the interior space to provide electrical power to the air treatment components. The case may receive at least part of the housing, which may be removably stored by the case.

In another aspect, the power component may further include a battery, power delivery electronics, and battery charging electronics. The battery may reversibly store the electrical power. The power delivery electronics may transmit the electrical power from the battery to the air treatment components. The battery charging electronics may receive external power from an external power source, condition the external power to a charging power receivable by the battery, and store at least part of the charging power in the battery for use by the air treatment components as the electrical power.

In another aspect, the housing may include a top housing portion, a bottom housing portion removably attached to the top housing portion, and housing inlets located on the bottom housing portion to permit the air to flow into the interior space. The fan component may receive the air from the housing inlets to be passed through the filtration component. The air that is passed through the filtration component may be moved to the nasal cavity.

In another aspect, the housing may include a locking interface comprising a locking interface top portion operatively attached to the top housing portion and a locking interface bottom portion operatively attached to the bottom housing portion. A locking member may be provided by the locking interface top portion to receive at least part of a locking lip of the locking interface bottom portion. A locking gap may be provided by the locking interface bottom portion having sufficient width to receive the locking member. The locking interface may be reversibly configured in a locked position by rotating the locking member passed through the locking gap in a first locking direction to receive the locking lip. The locking interface may be reversibly configured in an unlocked position by rotating the locking member in a second locking direction to release the locking lip.

In another aspect, the air treatment components may include a germicidal irradiation component to emit ultraviolet electromagnetic radiation that at least partially sterilizes pathogens from the air prior to entry into the nasal cavity.

In another aspect, the air treatment components may include an aroma component comprising an aroma reservoir to reversibly hold an aromatic material having an aroma detectable by an olfactory nerve. The aroma may be at least partially transferred from the aromatic material into the air to be delivered to the nasal cavity.

In another aspect, the filtration component may include a high efficiency particulate absorbing (HEPA) filter. In another aspect, the filtration component may include a carbon filter.

In another aspect, the apparatus may include an olfactory substance delivery component capable of administering a deliverable substance.

In another aspect, the deliverable substance may include a pharmacological substance.

In another aspect, the case may include a case enclosure and a case lid. The case enclosure may include a case receiving slot to selectively receive the housing. The case lid may be removably installable to the case enclosure to configure the case between a case open state and a case closed state. Electrical contacts may be located within the case receiving slot to interface with the power component. The power component may further include a battery to reversibly store the electrical power received from the case for use by the air treatment components. The housing may be selectively inserted into the case receiving slot or selectively removed from the case receiving slot when the case is configured in the case open state. Such housing that is received by the case receiving slot is substantially enclosed by the case while the case is configured in the case closed state.

In another aspect, a monitoring component may be provided that includes a sensor to detect a condition of the air included by an environmental space adjacent to the sensor. The condition is reportable via a communicably connected display device.

According to an alternative embodiment, the disclosure may feature an air purification apparatus for nasal application that includes a housing, a nose plug component, air treatment components, a power component, and a case. The housing may substantially enclose an interior space. The nose plug component may be operatively attached to the housing to be removably inserted into a nostril such to form an at least partial seal with a nasal cavity. The air treatment components may be located within the interior space to at least partially purify air for delivery to the nasal cavity. The air treatment components may include a germicidal irradiation component to emit ultraviolet electromagnetic radiation that at least partially sterilizes pathogens from the air prior to entry into the nasal cavity. The power component may be located substantially within the interior space to provide electrical power to the air treatment components. The housing may be selectively received by and selectively removed from a case.

In another aspect, the air treatment components may include a filtration component to remove impurities from the air and a fan component to actively pass the air through the filtration component.

In another aspect, the housing may include a top housing portion, a bottom housing portion removably attached to the top housing portion, and housing inlets located on the bottom housing portion to permit the air to flow into the interior space.

In another aspect, the power component may include a battery, power delivery electronics, and battery charging electronics. The battery may reversibly store the electrical power. The power delivery electronics may transmit the electrical power from the battery to the air treatment components. The battery charging electronics may receive external power from an external power source, condition the external power to a charging power receivable by the battery, and store at least part of the charging power in the battery for use by the air treatment components as the electrical power.

In another aspect, the air treatment components may include an aroma component with an aroma reservoir to reversibly hold an aromatic material having an aroma that is at least partially diffused into the air to be detectable by an olfactory nerve.

In another aspect, the filtration component may include a high efficiency particulate absorbing (HEPA) filter and a carbon filter.

In another aspect, the case may include a case enclosure comprising a case receiving slot to selectively receive the housing. The case may additionally include a case lid being removably installable to the case enclosure to configure the case between a case open state and a case closed state. Electrical contacts may be provided by the case to interface with the power component. The power component may further include a battery to reversibly store the electrical power received from the case for use by the air treatment components. The housing may be selectively inserted into the case receiving slot or selectively removed from the case receiving slot when the case is configured in the case open state. Such housing being received by the case receiving slot may be substantially enclosed by the case while the case is configured in the case closed state.

In another aspect, an olfactory substance delivery component is provided to administer a deliverable substance to the nasal cavity.

In another aspect, a monitoring component is provided with a sensor to detect a condition of the air included by an environmental space adjacent to the housing. The condition may be reported via a communicably connected display device.

Terms and expressions used throughout this disclosure are to be interpreted broadly. Terms are intended to be understood respective to the definitions provided by this specification. Technical dictionaries and common meanings understood within the applicable art are intended to supplement these definitions. In instances where no suitable definition can be determined from the specification or technical dictionaries, such terms should be understood according to their plain and common meaning. However, any definitions provided by the specification will govern above all other sources.

Various objects, features, aspects, and advantages described by this disclosure will become more apparent from the following detailed description, along with the accompanying drawings in which like numerals represent like components.

### BRIEF DESCRIPTION OF THE DRAWINGS

FIG. 1 is a perspective view of an illustrative air purification apparatus, according to an embodiment of this disclosure.

FIG. 2 is a perspective view of an illustrative air purification apparatus shown at an alternate angle with the housing installed, according to an embodiment of this disclosure.

FIG. 3 is a front elevation view of an illustrative air purification apparatus, according to an embodiment of this disclosure.

FIG. 4 is a rear elevation view of an illustrative air purification apparatus, according to an embodiment of this disclosure.

FIG. 5 is a bottom plan view of an illustrative air purification apparatus, according to an embodiment of this disclosure.

FIG. 6 is a top plan view of an illustrative air purification apparatus, according to an embodiment of this disclosure.

FIG. 7 is a cross-sectional view of an illustrative air purification apparatus shown in FIG. 3, according to an embodiment of this disclosure.

FIG. 8 is an exploded perspective view of an illustrative air purification apparatus, according to an embodiment of this disclosure.

FIG. 9 is a perspective view of an illustrative air purification apparatus included by a case aspect, according to an embodiment of this disclosure.

FIG. 10 is a partially exploded perspective view of an illustrative air purification apparatus included by a case aspect, according to an embodiment of this disclosure.

FIG. 11 is a perspective view of an alternative embodiment of an illustrative air purification apparatus, according to an embodiment of this disclosure.

FIG. 12 is a perspective view of an alternative embodiment of an illustrative air purification apparatus showing a bottom portion, according to an embodiment of this disclosure.

FIG. 13 is a front elevation view of an alternative embodiment of an illustrative air purification apparatus, according to an embodiment of this disclosure.

FIG. 14 is a bottom plan view of an alternative embodiment of an illustrative air purification apparatus, according to an embodiment of this disclosure.

FIG. 15 is a top plan view of an alternative embodiment of an illustrative air purification apparatus, according to an embodiment of this disclosure.

FIG. 16 is a cross-sectional view of an alternative embodiment of an illustrative air purification apparatus shown in FIG. 13, according to an embodiment of this disclosure.

FIG. 17 is an exploded perspective view of an alternative embodiment of an illustrative air purification apparatus, according to an embodiment of this disclosure.

FIG. 18 is a partially exploded perspective view of an alternative embodiment of an illustrative air purification apparatus showing a replaceable filter, according to an embodiment of this disclosure.

FIG. 19 is a front perspective view of a third embodiment of an illustrative air purification apparatus, according to an embodiment of this disclosure.

FIG. 20 is a front elevation view of a third embodiment of an illustrative air purification apparatus, according to an embodiment of this disclosure.

FIG. 21 is a top plan view of a third embodiment of an illustrative air purification apparatus, according to an embodiment of this disclosure.

FIG. 22 is an exploded view of a third embodiment of an illustrative air purification apparatus, according to an embodiment of this disclosure.

FIG. 23 is an exploded view of an alternative configuration of the embodiment shown in FIG. 22, according to an embodiment of this disclosure.

FIG. 24 is a perspective view of an illustrative use of an air purification apparatus enabled by this disclosure, according to an embodiment of this disclosure.

FIG. 25 is a perspective view of an illustrative air purification apparatus including a case configured in a case closed state, according to an embodiment of this disclosure.

FIG. 26 is a perspective view of an illustrative air purification apparatus including a case configured in a case open state, according to an embodiment of this disclosure.

FIG. 27 is an exploded view of an illustrative air purification apparatus shown in FIG. 25 including a case having a case lid, according to an embodiment of this disclosure.

### DETAILED DESCRIPTION

The following disclosure is provided to describe various embodiments of an air purification apparatus for nasal application. Skilled artisans will appreciate additional embodiments and uses of the present invention that extend beyond the examples of this disclosure. Terms included by any claim are to be interpreted as defined within this disclosure. Singular forms should be read to contemplate and disclose plural alternatives. Similarly, plural forms should be read to contemplate and disclose singular alternatives. Conjunctions should be read as inclusive except where stated otherwise.

Expressions such as “at least one of A, B, and C” should be read to permit any of A, B, or C singularly or in combination with the remaining elements. Additionally, such groups may include multiple instances of one or more element in that group, which may be included with other elements of the group. All numbers, measurements, and values are given as approximations unless expressly stated otherwise.

For the purpose of clearly describing the components and features discussed throughout this disclosure, some frequently used terms will now be defined, without limitation. The term HEPA filter, as it is used throughout this disclosure, is defined as a high-efficiency particulate absorbing filter being, using, or containing a filter usually designed to capture a substantial portion of airborne particles, for example 99.97 percent of airborne particles measuring 0.3 micrometers or greater in diameter, from air passing through the filter. The term ultraviolet (UV) light, as it is used throughout this disclosure, is defined as light with wavelengths shorter than that of visible light and longer than that of X-rays, including UV-C short-wavelength ultraviolet light that may be used for ultraviolet germicidal irradiation. The term nasal, as it is used throughout this disclosure, is defined as pertaining to the nose and related features. The term nostril, as it is used throughout this disclosure, is defined as an opening to a nasal cavity.

Various aspects of the present disclosure will now be described in detail, without limitation. In the following disclosure, an air purification apparatus for nasal application will be discussed. Those of skill in the art will appreciate alternative labeling of the air purification apparatus for nasal application as a nose filter device, nasal filtration device, portable powered air purification apparatus, the invention, or other similar names. Similarly, those of skill in the art will appreciate alternative labeling of the air purification apparatus for nasal application as an air filtration operation, irradiation and/or filtration technique, air quality monitoring and improvement method, method, operation, the invention, or other similar names. Skilled readers should not view the inclusion of any alternative labels as limiting in any way.

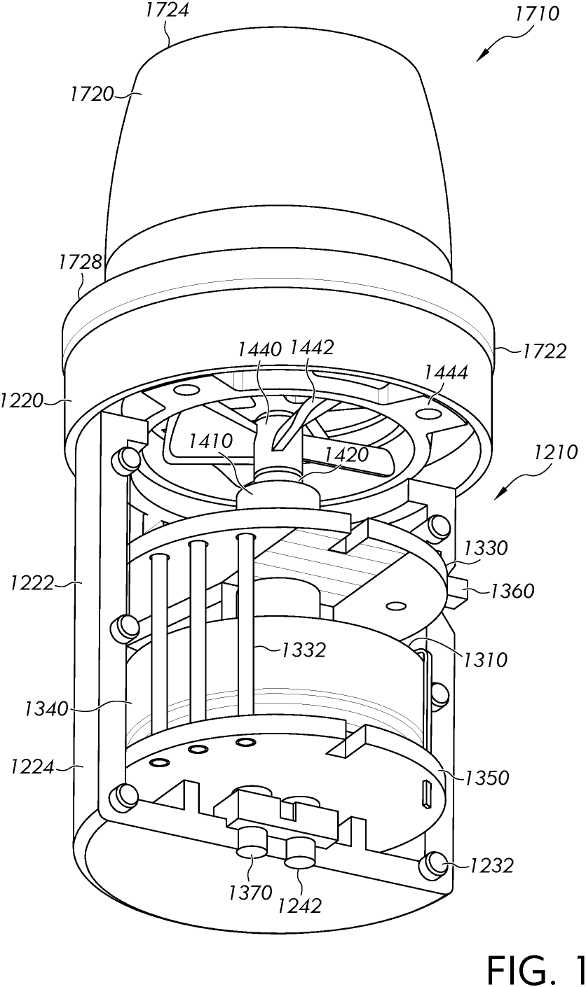

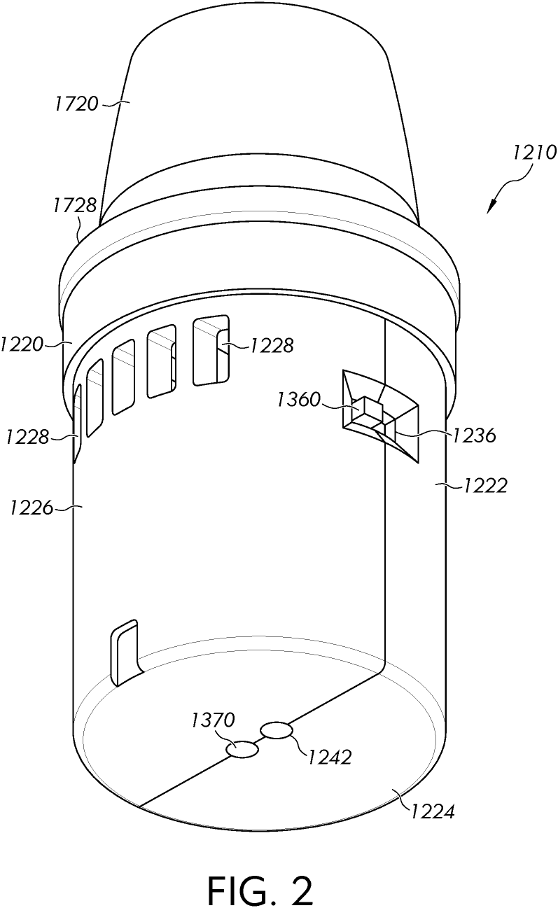

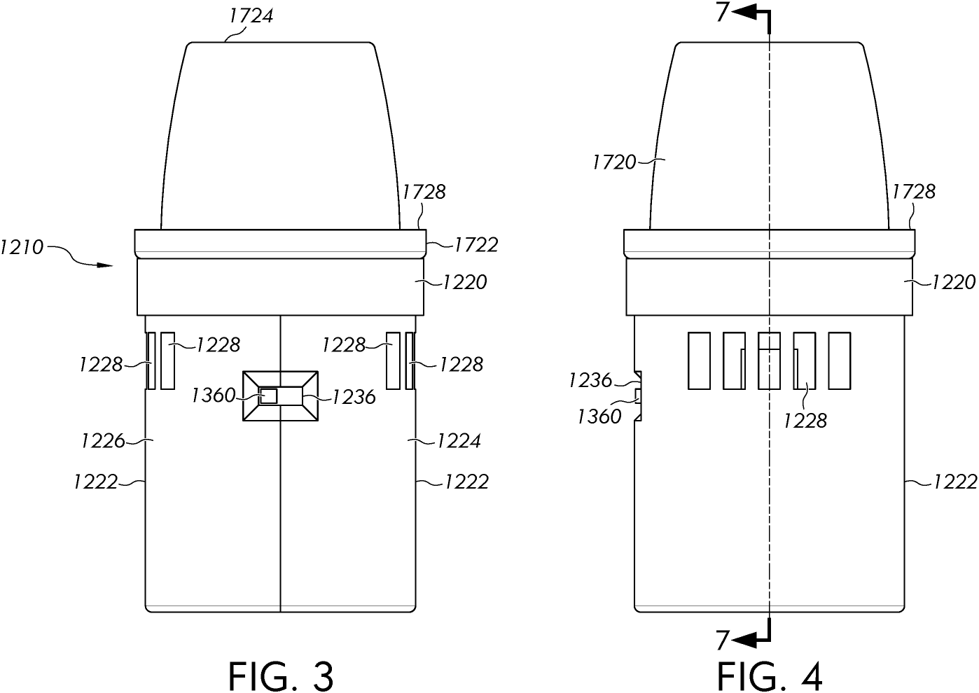

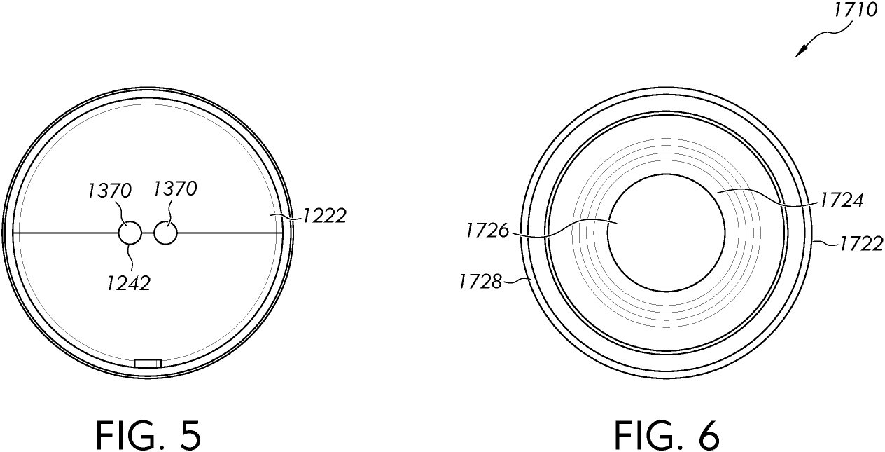

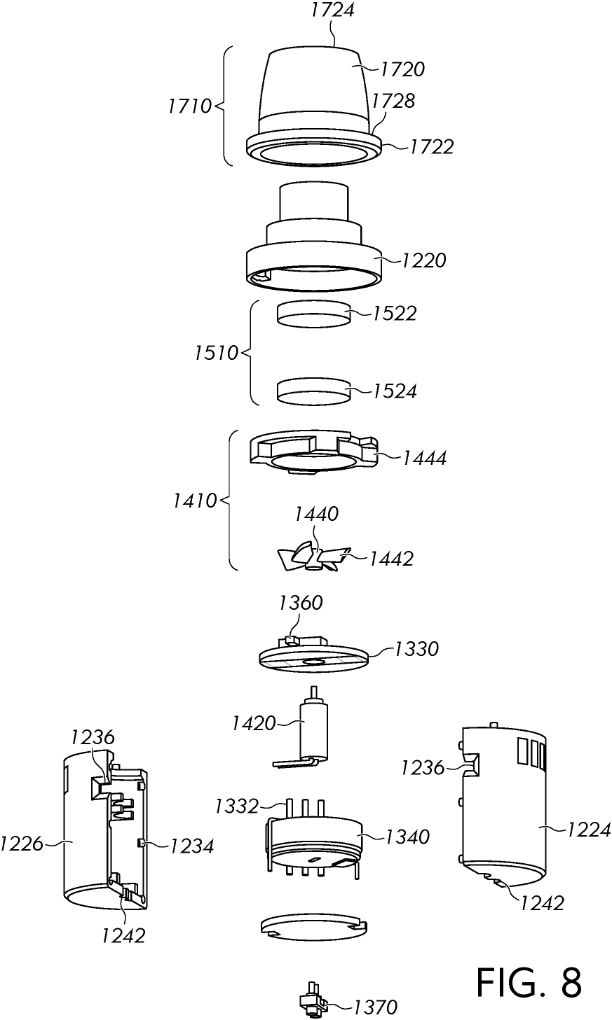

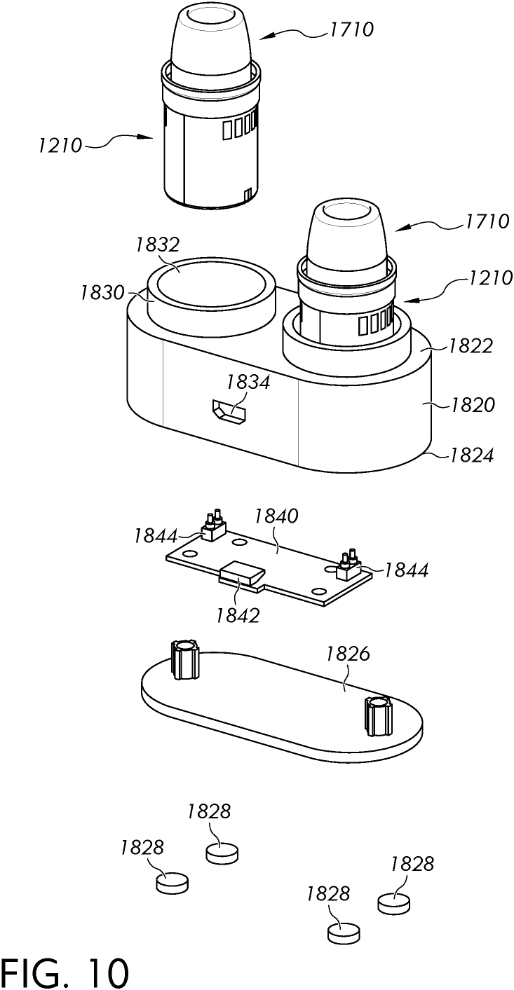

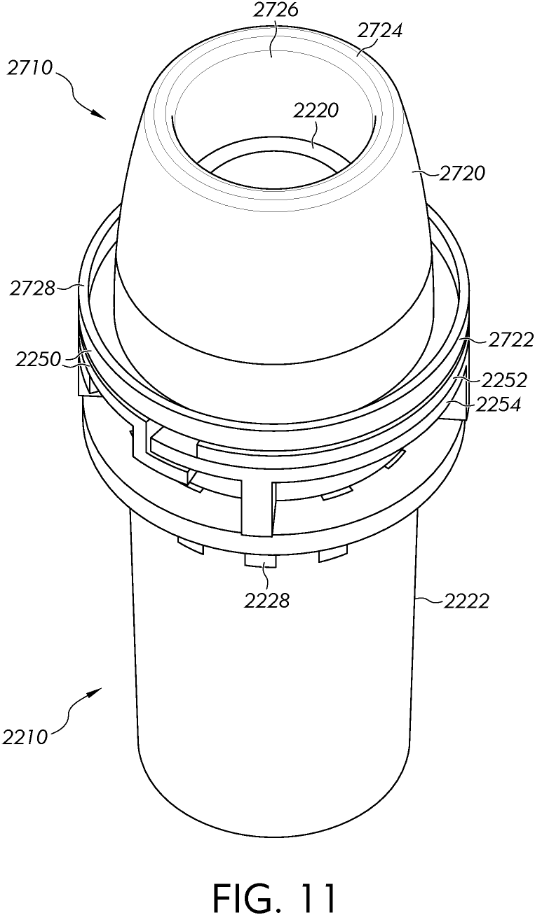

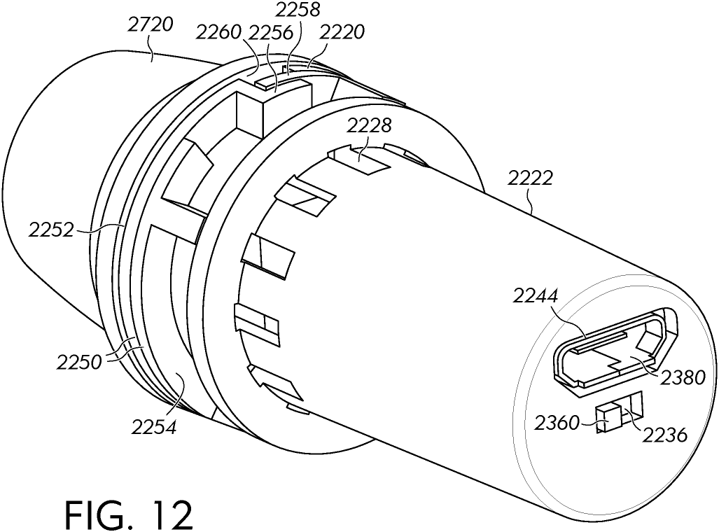

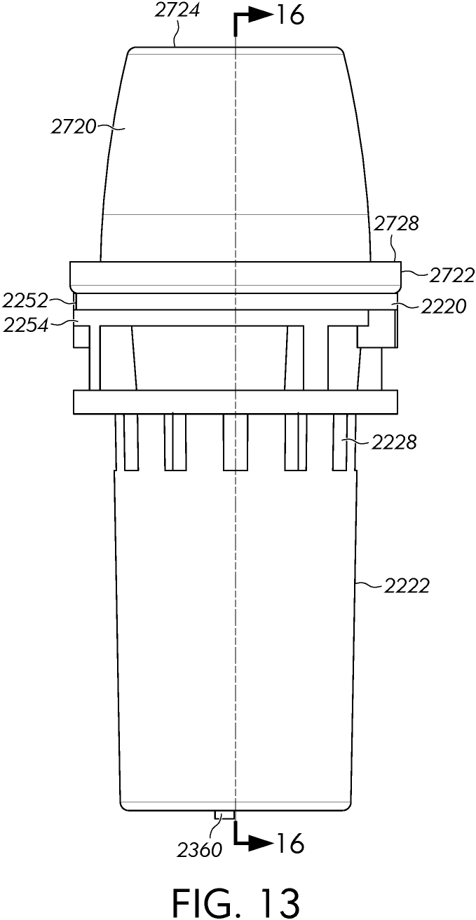

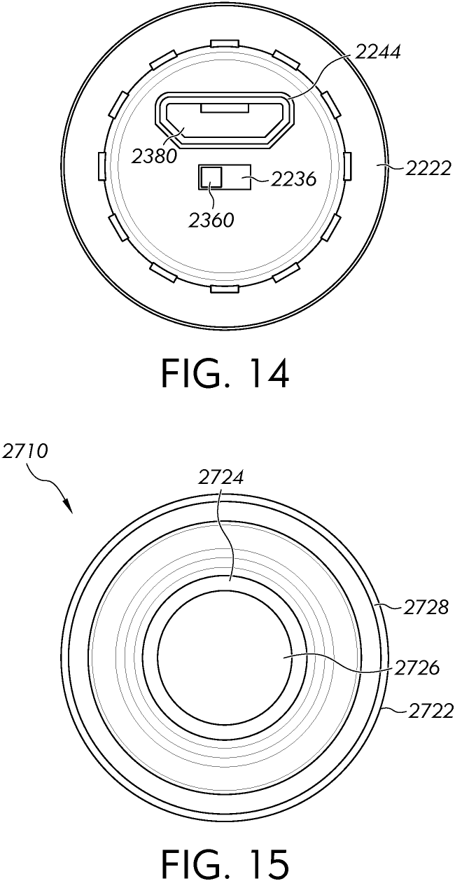

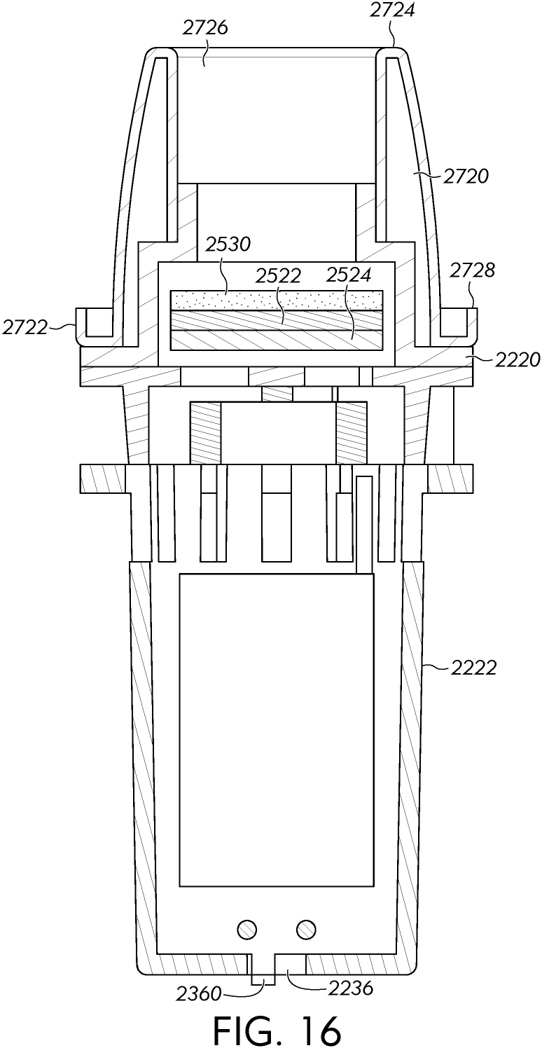

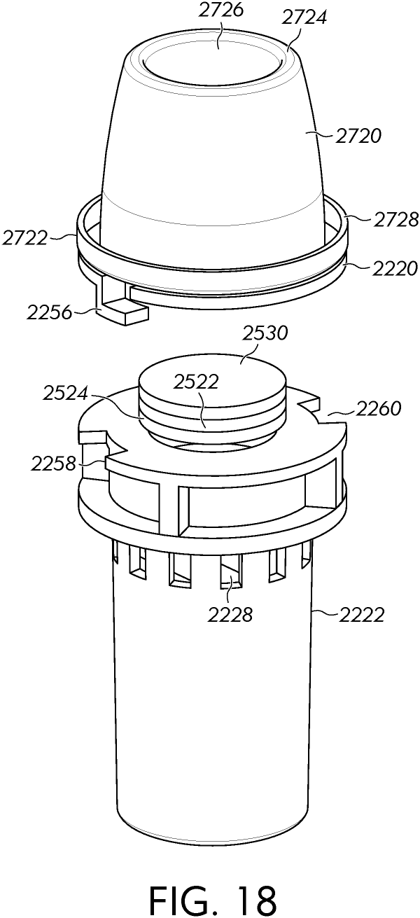

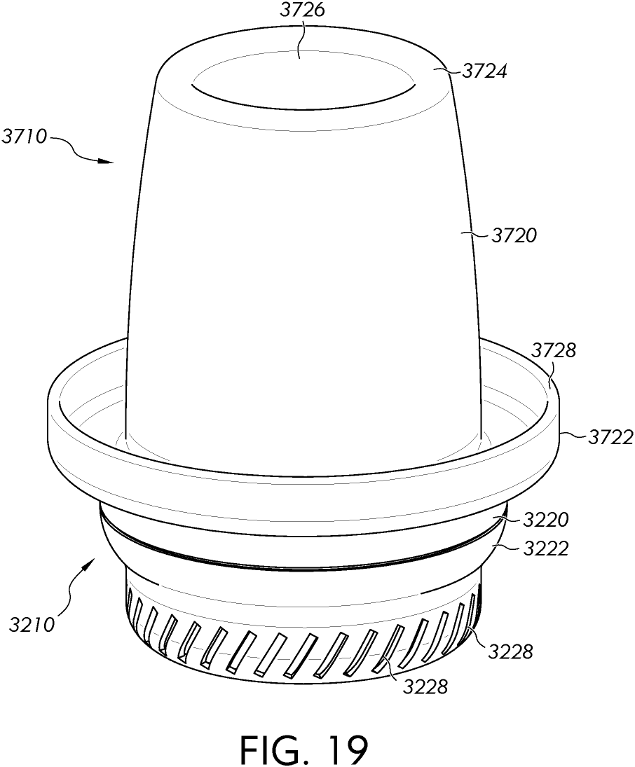

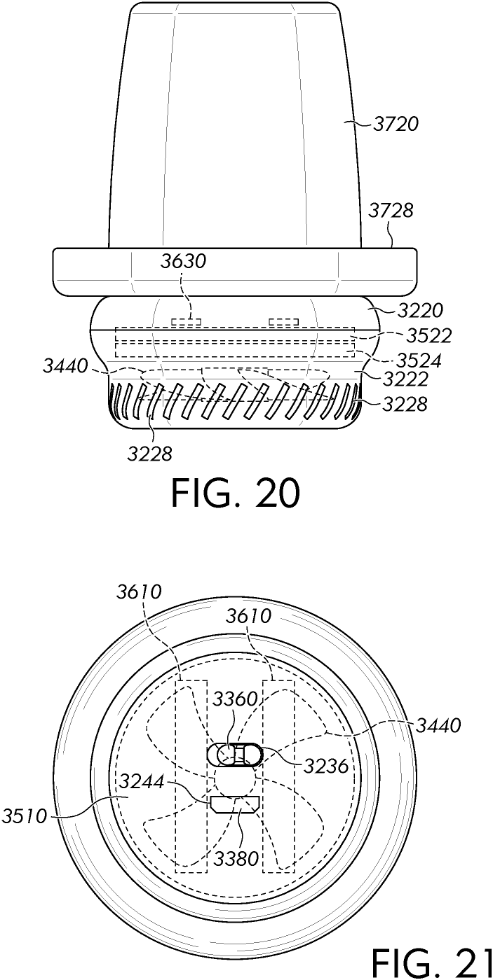

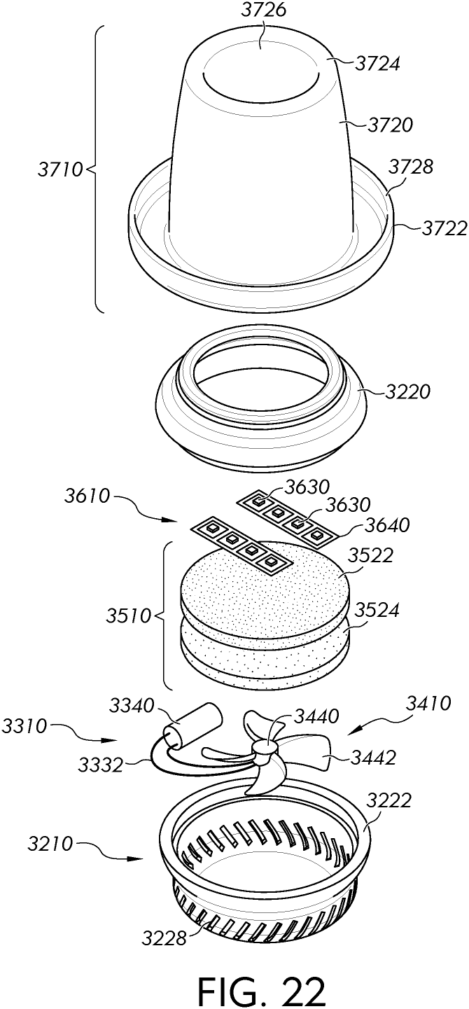

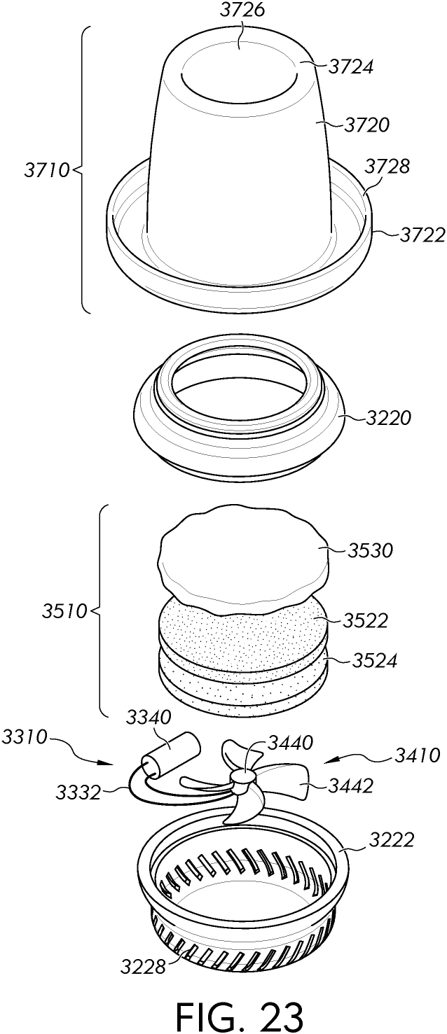

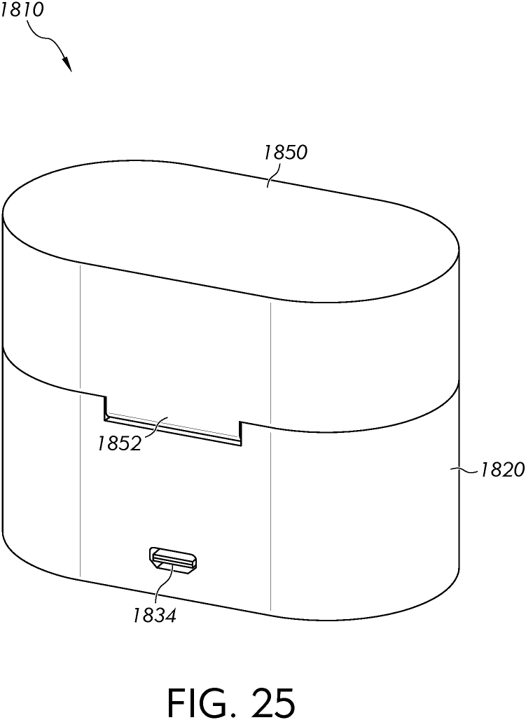

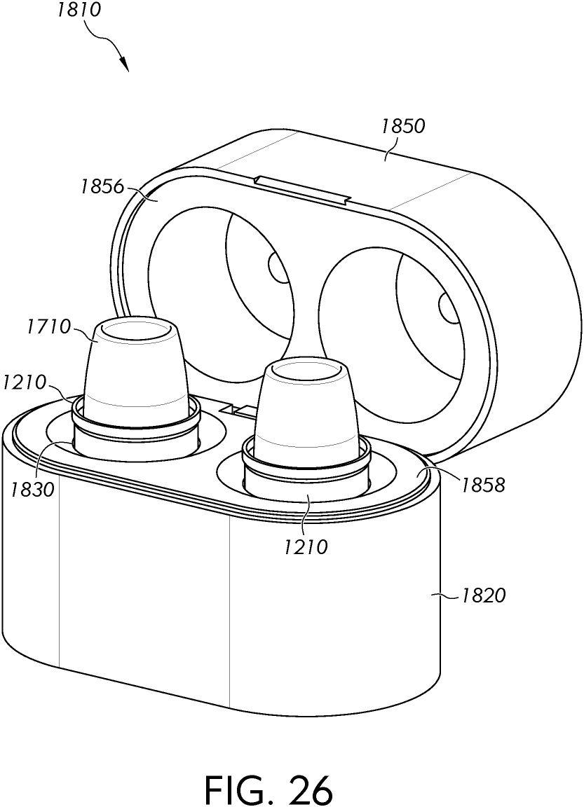

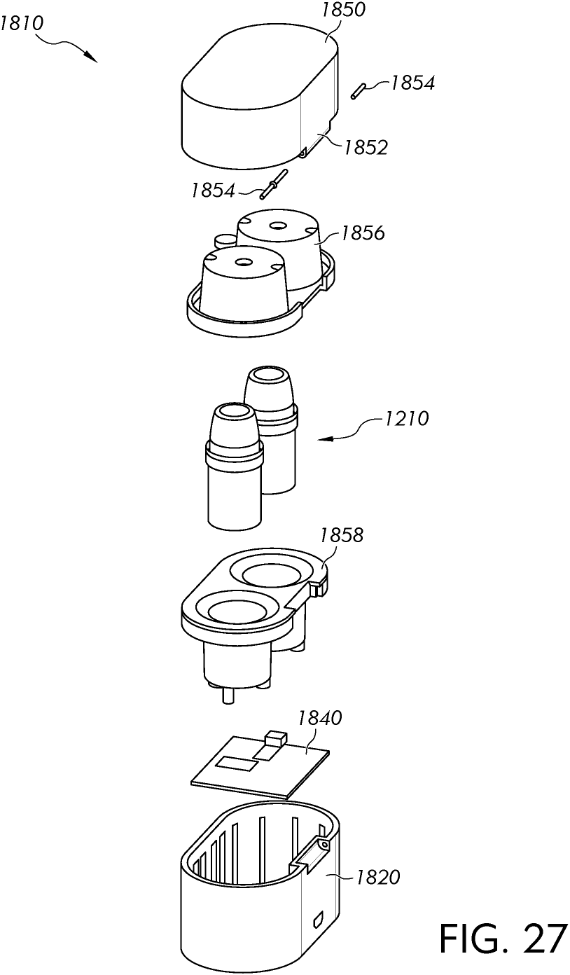

Referring now to FIGS. 1-27, an air purification apparatus for nasal application will now be discussed in more detail. The air purification apparatus for nasal application may include a housing, power component, fan component, filtration component, germicidal irradiation component, aroma component, olfactory substance delivery component, nose plug component, monitoring component, case, and additional components that will be discussed in greater detail below. The air purification apparatus for nasal application may operate one or more of these components interactively with other components for purifying and/or reducing undesirable materials from air intended to enter a nasal cavity.

In the interest of clearly presenting the invention, the following disclosure will be collectively discussed in the context of various illustrative embodiments. Skilled artisans should appreciate that features discussed in the context of one embodiment may be included in additional embodiments, even if not expressly stated when discussing such embodiments, and thus should view the teachings of this disclosure as a whole and not as a collection of disparate embodiments or otherwise limiting.

The air purification apparatus will now be discussed. FIGS. 1-27 highlight examples of the air purification apparatus in various embodiments. The air purification apparatus may include various components that collectively provide an improved capability to advantageously remove pathogens, contaminants, particulates, and/or other undesirable elements from air before it may enter the respiratory system of a user. In some embodiments, the air purification apparatus may include various components located within or attached to a housing. Such components may include, without limitation, air treatment components, a power component, a nose plug component, and other components. Air treatment components may include various aspects to improve the quality of air before it is received by a user, such aspects including without limitation, a filtration component, a fan component, a germicidal irradiation component, an aroma component, an olfactory substance delivery component, and/or other components that will be apparent to a person of skill in the art after having the benefit of this disclosure. Each of these components will be discussed in greater detail throughout this disclosure in the context of various embodiments. Skilled artisans should appreciate that each aspect may be included by alternative embodiments.

The housing will now be discussed in greater detail. FIGS. 1-5, 7-10, 11-14, 16-24, and 26-27 highlight examples of the housing, which may also be shown in other figures. The housing 1210, 2210, 3210 may be provided to substantially enclose an interior space within an air purification apparatus enabled by this disclosure. The housing 1210, 2210, 3210 may provide an enclosure to at least partially surround some of the functional components of an air purification apparatus within an interior space defined within the housing.

In an illustrative structural configuration, the housing 1210, 2210, 3210 may be at least partially comprised of plastic, without limitation. The housing may have virtually any diameter and height such to preserve operability of air treatment components. In one embodiment, the housing may have a substantially cylindrical shape, for example, between approximately 10 millimeters and approximately 30 millimeters in diameter, without limitation. The housing may have a height sufficient to enclose the air treatment components, for example, between approximately 7 millimeters and approximately 14 millimeters, without limitation.

The cylindrical shape may advantageously correspond with a nostril of a user for improved comfort, fit, and seal upon insertion into the nostril. Additionally, the use of a cylindrical shape may advantageously assist with the removal of an air purification apparatus enabled by this disclosure when use of such apparatus is no longer desired. In additional embodiments, the housing may be provided in a non-cylindrical shape, without limitation. For example, at least part of the housing may be provided by a rectangular shape, conical shape, rounded shape, and/or another shape that would be appreciated by those of skill in the art after having the benefit of this disclosure. In the example of a rectangular shape, the housing may be between approximately 10 millimeters and approximately 30 millimeters in width and between approximately 7 millimeters and approximately 14 millimeters in height, without limitation.

The housing 1210, 2210, 3210 may be constructed using various parts, for example a top housing portion 1220, 2220, 3220, a bottom housing portion 1222, 2222, 3222, and/or other portions that will be apparent after having the benefit of this disclosure. The housing 1210, 2210, 3210 may be sized with sufficient interior space to receive, hold, and allow operation of additional components including power components 1310, 2310, 3310, air treatment components, and/or other components that may be included within the interior space.

The housing 1210, 2210, 3210 may include access points to allow interaction with the components included within the interior space of the housing from an exterior of the housing. The access points may include a housing switch access point 1236, 2236, 3236 to allow access with a switch 1360, 2360, 3360, such as may be provided by the power component 1310, 2310, 3310. The access points may additionally include housing charging port access points 2244, 3244 to provide interaction with charging plugs and/or housing electrical contact access points 1242 to facilitate interaction with contact pins, each of which may provide electrical charge to aspects of the power component 1310, 2310, 3310 and/or additional components held within the interior space of the housing 1210, 2210, 3210.

In some embodiments, additional access points may be provided to enable additional functionality of an air purification apparatus enabled by this disclosure. Such additional access points may include sensor access points, display access points, points to access additional interactive elements related to the air treatment components and/or the power components, and otherwise.

The housing 1210, 2210, 3210 may additionally include housing inlets 1228, 2228, 3228 through which air may pass from an exterior location respective to the housing to the interior space substantially enclosed by the housing. For example, the housing 1210, 2210, 3210 may include numerous housing inlets 1228, 2228, 3228 at a bottom location with respect to the housing, such as near the lower end of the bottom housing portion 1222, 2222, 3222. This configuration may be advantageous wherein the internal space is organized such that air may pass through aspects of the power components 1310, 2310, 3310 without substantial restriction and be received by additional air treatment components before being presented to the nasal cavity of a user.

In another embodiment, the housing inlets 1228, 2228, 3228 may be provided near an upper location of the bottom housing portion 1222, 2222, 3222. This configuration may be advantageous wherein the interior space is organized such that the lower part of the bottom housing portion 1222, 2222, 3222 is substantially filled with power components 1310, 2310, 3310 such that airflow is maximized by entering the interior space of the housing 1210, 2210, 3210 above the location at which the power components are stored.

The housing inlets 1228, 2228, 3228 may be configured at various angles, which may affect the flow characteristics of the air entering the interior space of the housing 1210, 2210, 3210 from an exterior location. For example, the housing inlets 1228, 2228, 3228 may be configured with an angled orientation, which may improve the flow characteristics of air circulating within the interior space of the housing 1210, 2210, 3210, such as may be moved by a fan component 1410, 3410. In other embodiments, the housing inlets 1228, 2228, 3228 may be provided in substantially vertical orientation, which may advantageously provide flow characteristics to improve circulation for alternative fan configurations that would be apparent to those of skill in the art after having the benefit of this disclosure. Skilled artisans will appreciate additional configurations of housing inlets 1228, 2228, 3228 including horizontal, curved, angled, slotted, mixed, and otherwise configured orientations, without limitation.

In some embodiments, the bottom housing portion 1222 may be provided in multiple pieces. For example, the bottom housing portion 1222 may be provided having a bottom housing portion first piece 1224 and a bottom housing portion second piece 1226. The bottom housing portion first piece 1224 may be received by and substantially held by the bottom housing portion second piece 1226. For example, the bottom housing portion first piece 1224 may include one or more housing male connection members 1232 and the bottom housing second piece 1226 may include one or more housing female connection members 1234. The housing male connection members 1232 on the bottom housing portion second piece 1226 may be oriented such to correspond with the housing female connection members 1234 on the bottom housing portion second piece 1226. In this configuration, the housing male connection members 1232 may be inserted into the housing female connection members 1234 such to substantially hold both the bottom housing portion first piece 1224 and the bottom housing portion second piece 1226 together while coupled.

In some embodiments, the housing 1210 may include guideways to facilitate insertion of the housing 1210 into a case 1810. The case 1810 will be discussed in greater detail later in this disclosure. The guideways may advantageously assist the housing 1210 to be received by the case 1810 in a preferred orientation. For example, a case 1810 may be provided that includes electrical contacts 1844 capable of providing electrical power to an air purification apparatus enabled by this disclosure with corresponding electrical contacts 1370. In this example, the guideways may assist with orienting the housing 1210 such as to align the case 1810 and apparatus charging contacts to permit the transfer of electrical power. In additional embodiments, the guideways may include features to assist with improving a secure reception of the air purification apparatus enabled by this disclosure within the case 1810 upon insertion.

In another embodiment, the bottom housing portion 1222, 2222, 3222 may include a housing switch access point 1236, 2236, 3236 and/or a housing charging port access point 2244, 3244 upon its bottom surface. In this embodiment, a switch 1360, 2360, 3360 and/or charging port 2380, 3380 provided by the power component 1310, 2310, 3310 may be at least partially passed through the access points such that they may be interacted with by a user. For example, wherein a housing switch access point 1236, 2236, 3236 is included, a switch 1360, 2360, 3360 provided by the power component 1310, 2310, 3310 may be passed through the housing switch access point 1236, 2236, 3236 such that sufficient material of the switch 1360, 2360, 3360 is accessible for manipulation by a user. In another example, wherein a housing charging port access point 2244, 3244 is provided, a charging port 2380, 3380 may be at least partially passed through the house charging port 2380, 3380 access point such that it may receive a charging cable and/or other cable that may permit both the transfer of electrical power and/or data.

The housing 1210, 2210, 3210 may additionally include an interface to receive and/or hold a nose plug component 1710, 2710, 3710, which may be distal to the bottom surface of the housing. The nose plug component 1710, 2710, 3710 will be discussed in greater detail later in this disclosure. In this embodiment, the housing 1210, 2210, 3210 may include features to apply friction to the nose plug component 1710, 2710, 3710, textured aspects or protrusions to receive corresponding depressions on an interior surface of the nose plug component 1710, 2710, 3710, or otherwise be configured to operatively hold the nose plug component 1710, 2710, 3710 to the housing 1210, 2210, 3210 in a removably installed configuration.

In some embodiments, the housing 2210 may include a locking interface 2250. In some examples of an embodiment with a locking interface, the locking interface 2250 may serve as at least part of the housing 2210, such as being included by the top housing portion 2220. In other examples of an embodiment with a locking interface, the locking interface 2250 may be distinct and separate from the top housing portion 2220. Those of skill in the art will appreciate the inclusion of these various examples as nonlimiting.

The locking interface 2250 may include a locking interface top portion 2252 and a locking interface bottom portion 2254. The locking interface top portion 2252 may include a locking member 2256 that protrudes downward such as to be received by the locking interface bottom portion 2254. The locking interface bottom portion 2254 may include a locking lip 2258 that runs at least partially around the circumference of the locking interface bottom portion 2254. The locking lip 2258 may include a locking gap 2260 with sufficient width such that the locking member 2256 of the locking interface top portion 2252 may be passed through the locking gap 2260.

The distance between the lower portion of the locking member 2256 may be configured such that, once passed through the locking gap 2260, it may be rotated in a first locking direction to receive the locking lip 2258 and configure the locking interface 2250 in a locked position. Once configured in the locked position, the locking member 2256 may be substantially restricted in latitudinal movement by its reception of the locking lip 2258 and substantially limit access to the interior space provided by the housing 2210.

Additionally, the locked position may be reversible by rotating the locking interface top portion 2252 in a second locking direction such as to release the locking lip 2258 from the locking member 2256 and configure the locking interface 2250 and an unlocked position. Once configured in the unlocked position, the locking member 2256 may be withdrawn through the locking gap 2260 to provide access to the interior space otherwise enclosed by the housing 2210.

In some embodiments, the locking interface top portion 2252 may include a ring of material that extends at least partially around the circumference of the locking interface top portion 2252. If included, the ring of material provided by the locking interface top portion 2252 may advantageously assist with inserting the locking member 2256 within the locking gap 2260 to a sufficient depth to correspond with the locking lip 2258.

The power component will now be discussed in greater detail. FIGS. 1-5, 7-8, 12-14, 16-17, 22-23, and 27 highlight examples of the power component, which may also be shown in other figures. The power component 1310, 2310, 3310 may be located substantially within the interior space to provide electrical power to the air treatment components. The power components 1310, 2310, 3310 may advantageously assist with the conditioning, storage, and provision of electrical power to the various components of an air purification apparatus enabled by this disclosure. For example, the power components 1310, 2310, 3310 may provide electrical power to the various air treatment components held within the interior space provided by the housing 1210, 2210, 3210. The power component 1310, 2310, 3310 may include power delivery electronics 1330, a battery 1340, 2340, 3340, battery charging electronics 1350, and other aspects to assist with the transmission and delivery of electrical power.

The power component 1310, 2310, 3310 may include a battery 1340, 2340, 3340. In some embodiments, the battery may be rechargeable. In other embodiments, the battery may be removable from the interior space and replaceable. The battery 1340, 2340, 3340 may be virtually any size that may fit within the housing 1210, 2210, 3210. For example, the battery may be substantially cylindrical without limitation. In one example configuration, the battery may be between approximately 14 millimeters and approximately 20 millimeters in length and between approximately 5 millimeters and approximately 8 millimeters in diameter, without limitation.

Those of skill in the art will appreciate various additional configurations of batteries that may be used with an air purification apparatus, such as one enabled by this disclosure. Illustrative batteries may include lithium-ion batteries, nickel metal hydride batteries, rechargeable batteries, replaceable batteries, and/or other battery types that would be appreciated by those of skill in the art, without limitation.

The power component 1310, 2310, 3310 may include battery charging electronics 1350. In one example, the battery charging electronics 1350 may receive external power from an external power source, condition the external power to a charging power receivable by the battery 1340, 2340, 3340, and store at least part of the charging power in the battery for use by the air treatment components as the electrical power. The power component 1310, 2310, 3310 may control the application of the charging power to the battery 1340, 2340, 3340 to improve the charge state of the battery while advantageously decreasing the likelihood that the battery should become overcharged. Skilled artisans will appreciate various configurations of battery charging electronics 1350 which are intended to be within the scope and spirit of this disclosure. Skilled artisans will appreciate additional battery charging electronics and other features may be included by the power component 1310, 2310, 3310 to monitor the charge state of the battery, number of charge cycles applied to the battery, number of discharge cycles applied to the battery, battery temperature, battery voltage, irregularities detected within or with regard to the battery, and other features that may be provided by the battery charging electronics, without limitation.

The power component 1310, 2310, 3310 may additionally include power delivery electronics 1330 to provide electrical power from the battery 1340, 2340, 3340 to various components included within the interior space of an air purification apparatus enabled by this disclosure. The power delivery electronics 1330 may advantageously control the flow of electrical power to the various treatment components that operate by using the electrical power. For example, the power delivery electronics 1330 may control the power state of a fan component 1410, 3410, the operational speed of the fan included by the fan component, operation of a UV light source 3630 included by a germicidal irradiation component 3610, the duty cycle of such UV light source 3630, such as if provided as an LED, and other power-related operational variables that would be appreciated by those of skill in the art after having the benefit of this disclosure.

The power component 1310, 2310, 3310 may include electrical wires 1332, 3332, which may facilitate the transmission of electrical power from the various aspects included by the power component. Skilled artisans will appreciate additional configurations in which the electrical wires 1332, 3332 may be at least partially replaced by electrical contacts 1370, electrical traces provided in a circuit board, and/or otherwise, without limitation.

The power component 1310, 2310, 3310 may include a switch 1360, 2360, 3360 that may be toggled by a user to enable or disable the application of electrical power to the various air treatment components included within the interior space of an air purification apparatus enabled by this disclosure. The switch 1360, 2360, 3360 may be toggled between various states, which may include without limitation and on state and an off state. In some embodiments, the switch 1360, 2360, 3360 may include additional states such as to control selective engagement of various air treatment components that rely on electrical power delivered by the power component 1310, 2310, 3310. In additional embodiments, the switch 1360, 2360, 3360 may be operable through sensors that detect touch. In other embodiments, the switch may be provided as a continuous selection of electrical power application, as will be appreciated by those of skill in the art. In various embodiments, the switch 1360, 2360, 3360 and other power control mechanisms may be located virtually anywhere with respect to the housing 1210, 2210, 3210 if such placement does not interfere with the operability of air treatment components.

The power component 1310, 2310, 3310 may include interfaces to allow the battery charging electronics 1350 to receive the external power from the external power source. In one example, such interfaces may include electrical contacts 1370. In this example, the electrical contacts 1370 may be configured to orient with corresponding electrical contacts from the external power source, for example, a case 1810 having case electrical contacts 1844. In this example, once aligned, the electrical contacts 1370, 1844 may permit the flow of the external electrical power from the external power source through the electrical contacts 1370 and to the battery charging electronics 1350 before being stored in the battery 1340.

In another example, the interface allowing the battery charging electronics to receive the external power may include a charging port 2380, 3380. In some embodiments, the charging port 2380, 3380 may be provided as a USB-based port. Examples of USB-based ports may include USB Type-A, USB Type-B, micro USB, USB Type-C, and other USB standards that would be apparent to those of skill in the art. Additionally, those skilled in the art will appreciate additional plugs and corresponding ports capable of delivering electrical power that are intended to be included within the scope of the disclosure, without limitation.

In some embodiments, the charging port 2380, 3380 and corresponding charging cable may be configured to enable data transmission in addition to transmission of external power. In this embodiment, the functionality of an air purification apparatus enabled by this disclosure may be controlled, modified, updated, or otherwise affected depending on the data transmitted through the charging port 2380, 3380. In another example, a monitoring component provided by an air purification apparatus enabled by this disclosure may transmit data from such an air purification apparatus to an external computing device such as to perform analytics on conditions sense by sensors, operational logs, battery health, and/or other information that may be detected, stored, and made available by a monitoring component and/or other aspects of an air purification apparatus enabled by this disclosure. The data transmission may establish wired connectivity with a separate electronic device, such as a console capable of analyzing information transmitted to it regarding presence or absence of virus in a user utilizing an air purification apparatus such as, without limitation, presence, or absence of COVID-19 virus.

The air treatment components will now be discussed generally. Various components included by the air treatment components will be discussed in greater detail throughout this disclosure. Air treatment components may interact with air flowing through an interior space of an air purification apparatus, such as one enabled by this disclosure, to change a characteristic of such air. For example, the air treatment components may include a filtration component 1510, 2510, 3510 to remove particulates, pathogens, and other undesirable elements from the air as it passes through the interior space. In another example, a germicidal irradiation component 3610 may at least partially sterilize aspects of the air passing through the interior space. These and other components included by the air treatment components will be discussed in greater detail below.

The fan component will now be discussed in greater detail. FIGS. 1, 7-8, and 20-23 highlight examples of the fan component, which may also be shown in other figures. The fan component 1410, 3410 may be provided to assist with the movement of air throughout the interior space of an air purification apparatus, such as one enabled by this disclosure, during operation. The fan component 1410, 3410 may include a motor 1420, a fan 1440, 3440 having fan blades 1442, 3442, and optionally a fan shroud 1444. The motor 1420 may be operatively attached to the power delivery electronics 1330 and/or the battery 1340, 2340, 3340 to receive electrical power that may be used to drive the motor 1420. The speed at which the motor 1420 is operated may be at least partially controlled by the power delivery electronics 1330, for example by controlling the voltage applied to the motor, by controlling the frequency of pulses delivered to a pulse width modulation motor, or otherwise controlled as will be appreciated by those of skill in the art.

The fan 1440, 3440 may be oriented such as to draw air from the housing inlets 1228, 2228, 3228. This drawn air may be at least partially moved by the rotating fan blades 1442, 3442 provided by the fan 1440, 3440 and moved through, over, past, or otherwise in relation to other air treatment components included within the interior space. The fan component 1410, 3410 may advantageously provide active pressure within the interior space to direct the air through filters provided by a filtration component 1510, 2510, 3510, past UV light sources 3630 provided by a germicidal irradiation component 3610, aromatic material provided by an aroma component 2530, 3530, and/or otherwise as may be provided by the air treatment components included within the interior space of an air purification apparatus enable by this disclosure.

In one embodiment, the fan blades 1442 may be at least partially enclosed by a fan shroud 1444. In this embodiment, the fan shroud 1444 may assist with controlling the direction and movement of air passing by the fan blades. In some embodiments, the fan shroud 1444 may be included by at least part of the housing 1210. For example, the fan shroud 1444 may be built into at least part of the top housing portion 1220. In other embodiments, the fan shroud 1444 may be provided as a distinct member included within the interior space defined within the top housing portion 1220, 2220, 3220 and the bottom housing portion 1222, 2222, 3222. In some embodiments, the fan shroud 1444 may include an interior diameter such as corresponding with filters provided by the filtration component 1510, 2510, 3510. Those of skill in the art will appreciate additional advantageous applications of a fan shroud 1444 should it be included in such embodiments.

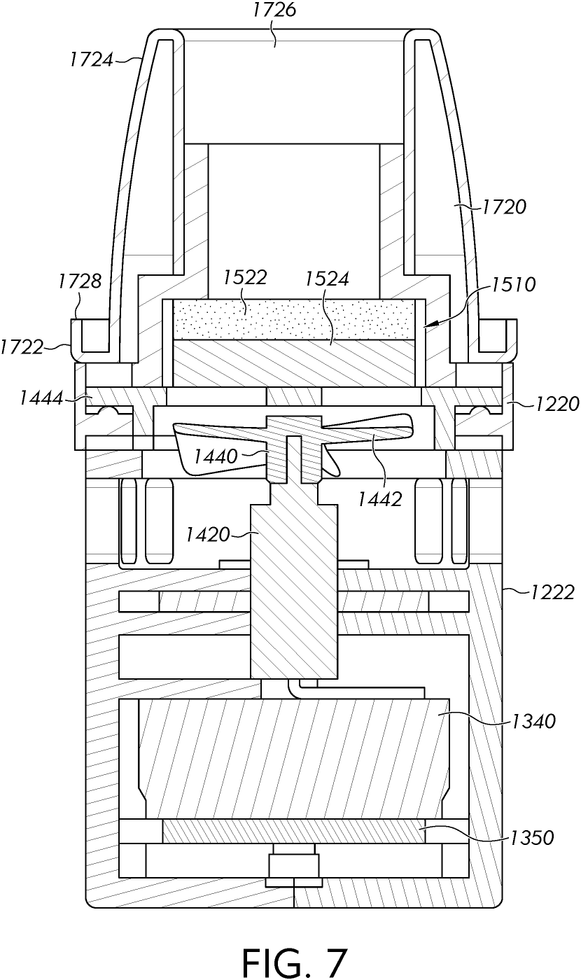

The filtration component will now be discussed in greater detail. FIGS. 7-8, 16-18, and 20-23 highlight examples of the filtration component, which may also be shown in other figures. The filtration component 1510, 2510, 3510 may include various filters and or other elements to affect the composition of air passing through or around the filtration component. In one embodiment, the filtration component 1510, 2510, 3510 may include a carbon filter 1524, 2524, 3524 to advantageously remove undesired particulates and other materials from air passing through such carbon filter. In one example, provided without limitation, a carbon filter may be provided a thickness of between approximately 5 millimeters and approximately 10 millimeters. The air may be passed through the carbon filter 1524, 2524, 3524 by active pressure applied by the fan component 1410, 3410 discussed above. In some embodiments, the carbon filter may include activated carbon, as will be appreciated by those of skill in the art.

The carbon filter 1524, 2524, 3524 may reduce the presence of airborne pathogens, including viruses and bacteria, from entering the respiratory system. The carbon filter 1524, 2524, 3524 may filter gaseous volatile organic compounds and other undesirable substances, such as cigarette smoke, drying paint, benzene, toluene, xylene, and/or other substances, without limitation. Carbon filtration may additionally filter unpleasant odors from air and may contribute to decreased stress and increased calmness and feelings of well-being on the part of such user. The carbon filter 1524, 2524, 3524 may include activated carbon.

In another embodiment, the filtration device may include a high efficiency particulate absorbing (HEPA) filter, as will be appreciated by those of skill in the art. In one example, provided without limitation, a HEPA filter may be provided having a wafer configuration with a thickness of between approximately 5 millimeters and approximately 10 millimeters. The HEPA filter 1522, 2522, 3522 may reduce potentially harmful agents and contaminants such as air bacteria, dust, pollen, mold, and other types of airborne particles. As will be appreciated by those of skill in the art, HEPA filtration may remove at least approximately 98% of airborne contaminants having a size of approximately 0.3 microns or more.

In some embodiments, either the carbon filter 1524, 2524, 3524 or the HEPA filter 1522, 2522, 3522 may be provided as the sole filtering technique included by the filtering component. In other embodiments, a two-stage filtering process may be provided whereby air is moved using the fan through the carbon filter 1524, 2524, 3524 for a first filtering pass and then through the HEPA filter 1522, 2522, 3522 for an additional filtering pass. In some embodiments, the filter may be scented or otherwise include and/or provide a substance capable of interacting with an olfactory nerve, without limitation. Those of skill in the art will appreciate additional filter types that may be included, which may also be arranged by the filtering component to affect the removal of particulates, pathogens, and other undesirable elements from the air passing through the filtration component 1510, 2510, 3510.

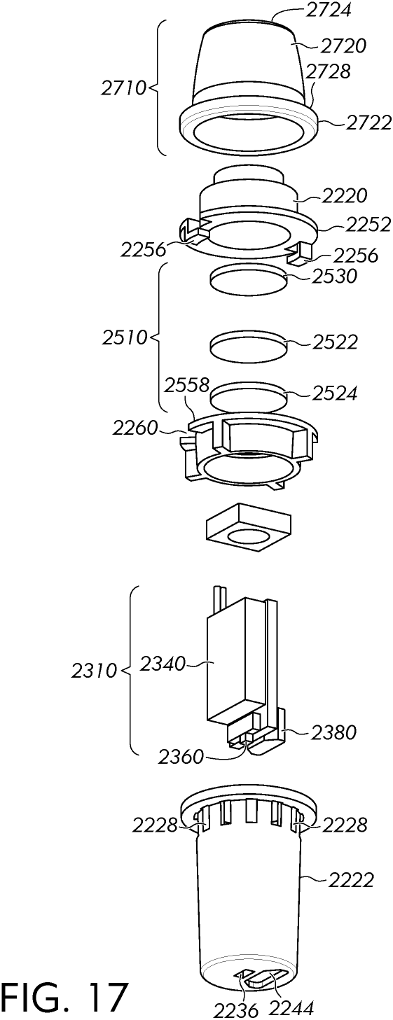

The aroma component will now be discussed in greater detail. FIGS. 17-19 and 24 highlight examples of the aroma component, which may also be shown in other figures. Inclusion of an aroma component 2530, 3530 may advantageously enhance a user’s feeling of well-being and calmness, thus decreasing a user’s stress. The aroma component may, without limitation, be located within a housing 2210, 3210 as contemplated herein. The aroma component may be located subsequent to the filtration component 2510, 3510, such as to receive the air after it has passed through the filtration component.

In one embodiment, an aroma component 2530, 3530 may be included such as to improve the aroma of the air passing through the interior space. The aroma may be detected by one or more olfactory nerves present in the nasal cavity and/or associated olfactory sensory organs. The aroma component 2530, 3530 may advantageously apply a pleasurable or otherwise desirable scent to the air, increasing the mood and psychological well-being of a user operating an air purification apparatus such as one enabled by this disclosure, for example, as the aroma interacts with one or more olfactory nerves. In another embodiment, the aroma component 2530, 3530 may provide essential oils, congestion relief solutions, and/or other substances that may be added to the air prior to being sent to the nasal cavity of the user.

The aroma component 2530, 3530 may include an aroma reservoir to hold an aromatic material capable of providing an aroma. An illustrative aroma reservoir may include porous materials, sponges, sponge-like materials, wafers, meshes, absorbent materials, liquid basins, and other materials or containers that would be apparent to a person of skill in the art. Without limitation, an illustrative aroma reservoir constructed as a porous material may have a thickness of between approximately 5 millimeters and approximately 10 millimeters. The aromatic material may be a perfume, essential oil, aromatic solution, natural ingredient, synthetic ingredient, and/or another material that would be appreciated by those of skill in the art after having the benefit of this disclosure.

The aroma component 2530, 3530 may be configured such that upon application of positive pressure by the fan component 1410, 3410, air passes through the interior space of an air purification apparatus such as one enabled by this disclosure sufficiently to come in contact with the aroma reservoir, thus transferring at least part of the aroma provided by the aromatic material to the air. The aromatic air may then pass through the nose component and into the nasal cavity of a user engaging the olfactory receptors and providing a psychological and/or satisfactory response to the user.

The olfactory substance delivery component will now be discussed in greater detail. Inclusion of an olfactory substance delivery component may advantageously assist with the provision of deliverable substances via operation of the air purification apparatus and/or the included air treatment components. For the purpose of this disclosure, a deliverable substance may include pharmacological substances, nutraceutical substances, holistic treatments, essential oils, nebulized liquids, and/or other substances that would be appreciated by a person of skill in the art after having the benefit of this disclosure. In some embodiments, the deliverable substances may include nanoparticles, which may include ultrafine particles that range between about 1 and 100 nanometers in diameter. Features may be provided to assist with delivering only a desired dosage of deliverable substances. In one example, provided without limitation, a pharmaceutical treatment for Alzheimer’s disease may be administered to a user via the olfactory substance delivery component.

The olfactory substance delivery component may, without limitation, be located within a housing 1210, 2210, 3210 as contemplated herein. The olfactory substance delivery component may be located subsequent to the filtration component 1510, 2510, 3510 and/or the aroma component 2530, 3530, such as to receive the air after it has passed through the filtration component, without limitation.

In one embodiment, an olfactory substance delivery component may be included such as to provide a deliverable substance to the nasal cavity and/or associated nasal spaces. The olfactory substance delivery component may include a deliverable substance reservoir to hold a deliverable substance capable of being administered to a user. An illustrative deliverable substance reservoir may include porous materials, sponges, sponge-like materials, wafers, meshes, absorbent materials, liquid basins, and other materials or containers that would be apparent to a person of skill in the art. Without limitation, an illustrative deliverable substance reservoir constructed as a porous material may have a thickness of between approximately 5 millimeters and approximately 10 millimeters. The deliverable substance may be a pharmaceutical, drug, nutraceutical, antiseptic solution, natural ingredient, synthetic ingredient, and/or another material that would be appreciated by those of skill in the art after having the benefit of this disclosure.

The olfactory substance delivery component may be configured such that upon application of positive pressure by the fan component 1410, 3410, air passes through the interior space of an air purification apparatus, such as one enabled by this disclosure, sufficiently to come in contact with the deliverable substance reservoir, thus transferring at least part of the deliverable substance provided by the olfactory substance delivery component to the air. The air carrying the deliverable substance may then pass through the nose component and into the nasal cavity of a user engaging the olfactory receptors and/or other receptors providing a medical, therapeutic, psychological, and/or otherwise satisfactory response to the user.

The germicidal irradiation component will now be discussed in greater detail. FIGS. 21-23 highlight examples of the germicidal irradiation component, which may also be shown in other figures. The germicidal irradiation component 3610 may emit ultraviolet electromagnetic radiation that at least partially sterilizes pathogens from the air prior to entry into the nasal cavity, such as hazardous microorganisms, bacteria, and viruses.

The germicidal irradiation component 3610 may be configured to emit electromagnetic energy capable of at least partially sterilizing materials included in the air passing through and contacting the electromagnetic energy. In the interest of simplicity in this disclosure, the electromagnetic energy will be discussed in the context of ultraviolet light, without limitation. Those of skill in the art will appreciate additional electromagnetic energy that may additionally provide disinfecting and/or germicidal benefits that are intended to be included within the scope of this disclosure.

The germicidal irradiation component 3610 may include a light source capable of emitting light having frequencies that facilitate the sterilization of undesired contaminants, pathogens, and other elements in the air passing by the germicidal irradiation component 3610. The light source may be an ultraviolet light source that may emit light in the frequency range within the ultraviolet spectrum, as will be appreciated by those having skill in the art. In some embodiments, the ultraviolet light source may include light emitting diodes (LEDs) 3630 that emit or otherwise provide light in the ultraviolet spectrum. In some embodiments, multiple ultraviolet light sources may be included on a substrate 3640, for example, a circuit board onto which the ultraviolet light sources 3630 are installed.

The ultraviolet light sources 3630, for example via the substrate 3640, may be powered at least partially by the power delivery electronics 1330 of the power component 3310. In some embodiments, the ultraviolet light source may be provided as a dimmable and/or otherwise controllable configuration. For example, the brightness and power apply to the ultraviolet light source may be at least partially controlled by affecting the duty cycle at which the light source operates. The duty cycle may be controlled by the power delivery electronics, other aspects included by the germicidal irradiation component 3610, and/or the power component 3310, without limitation.

In some embodiments, the germicidal irradiation component 3610 may include multiple substrates 3640, each of which including multiple ultraviolet light sources 3630 capable of emitting a sufficient amount of ultraviolet light such as to substantially sterilize contaminants included in the air passing by the germicidal irradiation component 3610. The intensity of light emitted by the ultraviolet light sources 3630 may be controlled with respect to the number of ultraviolet light sources included by the germicidal irradiation component 3610. For example, a number of high intensity ultraviolet light sources may be provided by the germicidal irradiation component 3610 to allow a high degree of sterilizing light to be emitted by selected number of ultraviolet light sources. In another example, a larger quantity of ultraviolet light sources may provide sterilizing light at a lower intensity, but collectively providing a sufficient amount of sterilizing light over a wider area of application from the larger number of ultraviolet light sources.

In some embodiments, the germicidal irradiation component 3610 may include additional light emitting sources and/or other electromagnetic energy emitting sources to further sterilize aspects of the air passing by the germicidal irradiation component 3610. Such light sources may include characteristics indicative of ultraviolet-C (UV-C) light, otherwise referred to as shortwave ultraviolet light, such to disrupt the ability of pathogens and other materials to replicate after exposure to the emitted ultraviolet light. Additional germicidal light sources will be appreciated by those of skill in the art after having the benefit of this disclosure.

The nose plug component will now be discussed in greater detail. FIGS. 1-1-4, 6-13, 15-20, 22-24, and 26-27 highlight examples of the nose plug component, which may also be shown in other figures. The nose plug component 1710, 2710, 3710 may be operatively attached to the housing 1210, 2210, 3210 to be removably inserted into a nostril such to form an at least partial seal with a nasal cavity. The nose plug component 1710, 2710, 3710 may be constructed of a durable material to minimize potential damage when they are being handled, which material being substantially nontoxic to humans, for example silicon or graphene.

The nose plug component 1710, 2710, 3710 may be operatively installed to at least part of the housing 1210, 2210, 3210 provided by an air purification apparatus enabled by this disclosure. The nose plug component 1710, 2710, 3710 may include a nose plug 1720, 2720, 3720 having a nose plug base end 1722, 2722, 3722 and a nose plug nostril end 1724, 2724, 3724. The nose plug 1720, 2720, 3720 may have a circumference and a diameter of a size such that, upon insertion into a nostril, the device may remain in place in the nostril anchored by friction. In one embodiment, the nose plug 1720, 2720, 3720 may have a diameter of between approximately 8 millimeters and approximately 14 millimeters and a length between approximately 15 millimeters and approximately 25 millimeters, without limitation.

The nose plug 1720, 2720, 3720 may include a nose plug aperture 1726, 2726, 3726 near the nose plug nostril end 1724, 2724, 3724. The nose plug 1720, 2720, 3720 may additionally include a nose plug protruding lip 1728, 2728, 3728 near the nose plug base end 1722, 2722, 3722. Additional features may be provided by the nose plug that would be appreciated by those of skill in the art after having the benefit of this disclosure.

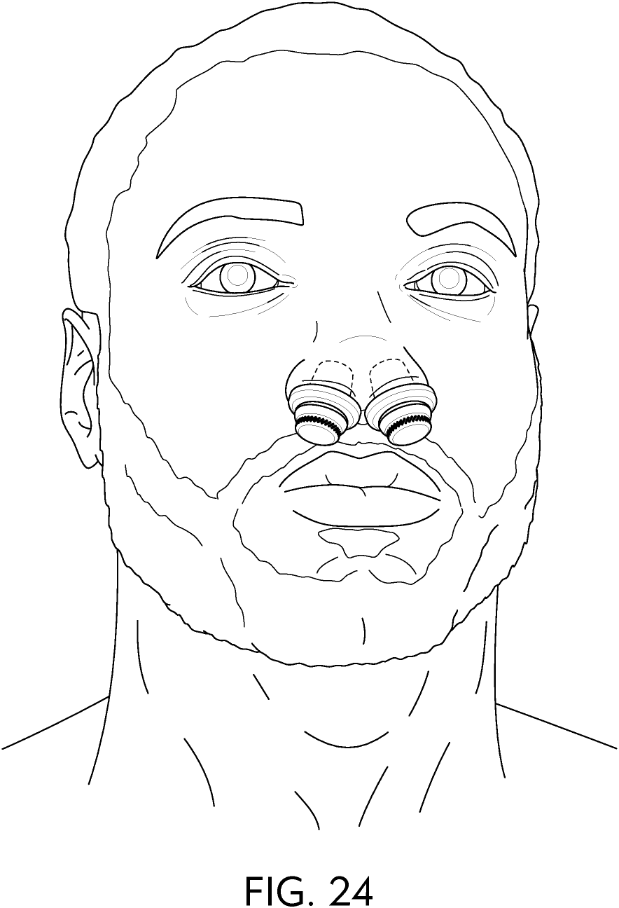

In one embodiment, provided without limitation, the nose plug 1720, 2720, 3720 may be constructed using an at least partially flexible material, for example silicone, such that it may be inserted into the nostril of a user with minimal or negligible irritation. Additionally, by using a flexible material, the nose plug may provide an at least partial seal within the nostril such as to provide access to the nasal cavity of a user upon operation. The nose plug 1720, 2720, 3720 may be configured such that the nose plug may be inserted at least partially into the nostril providing a pathway from the interior space provided by the housing 1210, 2210, 3210 through the nose plug 1720, 2720, 3720 and into the nasal cavity. Accordingly, when in use, such nose plug components 1710, 2710, 3710 may extend into a nostril to a depth of between approximately 15 millimeters and approximately 25 millimeters, without limitation, as illustrated in FIG. 24.

The nose plug 1720, 2720, 3720 may include a nose plug aperture 1726, 2726, 3726 that runs substantially throughout the interior of the nose plug. The nose plug aperture 1726, 2726, 3726 may extend substantially from the nose plug base end 1722, 2722, 3722 through the nose plug 1720, 2720, 3720 to the nose plug nostril end 1724, 2724, 3724. Air may be moved through the nose plug aperture 1726, 2726, 3726 actively by operation of the fan component 1410, 3410 discussed above, passively through respiration by a user, and/or otherwise as would be appreciated by those of skill in the art. In some embodiments, the nose plug aperture 1726, 2726, 3726 may include a mesh material capable of serving as an additional layer of filtration for any remaining undesired particulate matter that has not already been filtered out when air is moving through an air purification apparatus enabled by the present disclosure.

The nose plug component 1710, 2710, 3710 may include a nose plug protruding lip 1728, 2728, 3728, which may advantageously prevent the nose plug from being inserted undesirably deep within the nasal cavity of a user. The nose plug protruding lip 1728, 2728, 3728 may be substantially contiguous with the nose plug 1720, 2720, 3720 and may be constructed using the same materials as the nose plug. The nose plug protruding lip 1728, 2728, 3728 may advantageously provide a stopping mechanism, thus providing physical resistance from over-insertion of the nose plug 1720, 2720, 3720. In some embodiments, the nose plug protruding lip may have increased rigidity over the portion of the nose plug to be inserted into the nostril such to increase its stopping capability.

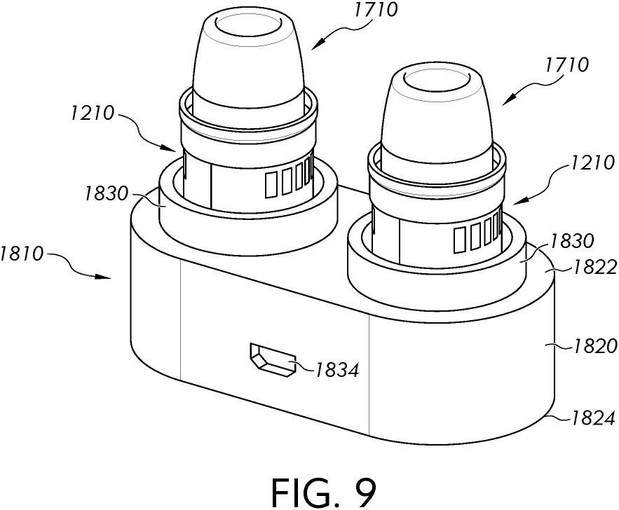

The case will now be discussed in greater detail. FIGS. 9-10 and 25-27 highlight examples of the case, which may also be shown in other figures. In some embodiments, a case 1810 may be provided to receive, store, charge, and otherwise interact with an air purification apparatus such as one enabled by this disclosure. For example, a case 1810 may be configured to receive an air purification apparatus when not in use such as when not being inserted into the nasal cavity of a user.

In one embodiment, the case 1810 may include a case enclosure 1820 which may extend from a case enclosure bottom end 1824 to a case enclosure top end 1822. The case enclosure 1820 may include case receiving slots 1830 at its case enclosure top end 1822 to receive and/or substantially secure the housing 1210 of an air purification apparatus enabled by this disclosure. The case enclosure receiving slot 1830 may include a case receiving slot aperture 1832 through which the housing 1210 may be inserted. The case enclosure receiving slot 1830 may include a protruding guideway which may interact with a depressed guideway included by the housing 1210 to assist with orienting the housing 1210 properly upon insertion into the case receiving slot 1830.

In some embodiments, the case 1810 may include a case lid 1850 that may be operatively attached to the case enclosure 1820 to collectively form the case 1810. The case lid 1850 may be operatively attached to the case enclosure 1820 such as to allow the case 1810 to be manipulated between a case closed state and a case opened state. In one example, the case lid 1850 may be operatively attached to the case enclosure 1820 via a case hinge 1852. In this example, the case lid 1850 may include a first portion of material that extends into a recessed second portion of material provided by the case enclosure 1820 such as to align pathways associated with the case hinge 1852 configured to receive case hinge pins 1854, as will be appreciated by those of skill in the art. This example may advantageously facilitate altering the case 1810 between the case closed state and the case opened state by pivoting the case lid 1850 about an axis aligned with the case hinge 1852 through which the case hinge pins 1854 are oriented.

In other embodiments, an alternative configuration may be used to removably attach the case lid 1850 to the case enclosure 1820. Example configurations include, without limitation, friction, snapping interfaces, magnetics, ribbing, latches, and/or other configurations that would be appreciated by those of skill in the art after having the benefit of this disclosure.

The case lid 1850 may include a case inner lid 1856, which may be at least partially installed into an interior space provided by the case lid 1850. Inclusion of a case inner lid 1856 may advantageously assist with securing the housing 1210 installed into to case 1810 via a case receiving slot 1830 while the case 1810 is configured in the case closed state. For example, the case inner lid 1850 may include sufficient clearance such to correspond with the nose plug component 1710 attached to the housing 1210 in a manner that holds the housing 1210 in place while the case lid 1850 is closed and the case 1810 is configured in the case closed state. Additionally, the case inner lid 1856 may be positioned as the case 1810 is configured in the case opened state to provide minimal interference with the housing 1210 held by the case 1810 to facilitate insertion of the housing into, and removal of the housing from, the case receiving slot 1830.

The case enclosure 1820 may include a case base 1858, which may be at least partially installed into an interior space provided by the case enclosure 1820. Inclusion of a case base 1858 may advantageously assist with securing the housing 1210 installed into to case 1810 by providing the case receiving slot 1830 to removably receive the housing 1210 and substantially secure the received housing 1210 while the case 1810 is configured in the case closed state. For example, the case base 1858 may include sufficient space such to correspond with the housing 1210 in a manner that holds the housing 1210 in place while the housing is inserted into the case receiving slot 1830. The housing 1210 may be held by the case base 1858 in both the case open state and case closed state, with the housing 1210 begin insertable and removable while the case 1810 is configured in the case opened state.

The case 1810 may include case power electronics 1840 to assist with charging or otherwise providing power to an air purification apparatus enabled by this disclosure when inserted into the case 1810. For example, the case 1810 may include case power electronics 1840 capable of receiving external electrical power from an external power source via a case charging port 1842, which may be accessible via a case charging port access point 1834. The case power electronics 1840 may condition and/or otherwise affect the external power into a charging power capable of being received by the electrical contacts 1370 provided by the power component 1310 of the air purification apparatus enabled by the disclosure. For example, upon insertion of the housing 1210 of the air purification apparatus into the case receiving slot 1830, electrical contacts 1370 provided by the air purification apparatus may come into operative connection with electrical contacts 1844 provided by the case 1810, which may be accessible via the case receiving slot 1830. Once contact is established, charging power may be provided by the case 1810 to the battery 1340 of the air purification apparatus. In some embodiments, the electrical charging circuitry of the air purification apparatus may interact with power received by the case 1810 and condition the power such that it is storable in the battery 1340 included by the power component 1310. In other embodiments, power may be delivered by the case 1810 such that it may be applied directly to the battery 1340 and thus eliminate the need for separate electrical charging circuitry within the air purification apparatus itself.

The case 1810 may include a case charging port 1842, which may be provided as a USB port and/or other port capable of carrying electrical power, such as discussed above in the context of the power component 1310. In some embodiments, the case charging port 1842 may enable transmission of data received from the air purification apparatus located within the case receiving slot 1830. The case 1810 may operate as a pass-through device providing access between the case charging port 1842 provided by the case 1810 and the components held within the interior space of the air purification apparatus. In other embodiments, active electronics may be included within the case 1810 that can at least partially condition the power, facilitate the transmission of data, and/or otherwise assist with the operation of the air purification apparatus received by the case receiving slot 1830.

The case 1810 may include a case bottom panel 1826 located at the case enclosure bottom end 1824. The case bottom panel 1826 may be removably installed to the case 1810, which may provide access to an interior volume of the case upon removal from the case. One or more case feet 1828 may optionally be provided to improve the stability of the case 1810 when located on a surface and/or reduce the likelihood of damage to a surface on which the case may be located.

In some embodiments, the case 1810 may be configured to receive multiple housings, which may be facilitated by providing multiple case receiving slots 1830. For example, a case may be configured to receive, hold, and/or charge six or eight air purification apparatuses. Those of skill in the art will appreciate additional embodiments of the case which may be configured to hold various number of housings and/or air purification apparatuses without limitation, for example being configured to hold 1, 2, 3, 4, 5, 6, 7, 8, 9, 10, 11, 12, 14, 16, 18, 20, or another number of housings and/or air purification apparatuses.

The monitoring component will now be discussed in greater detail. In one embodiment, a monitoring component may be provided that includes a sensor to detect a condition of the air included by an environmental space adjacent to the sensor. The condition may be reported via a communicably connected display device. The monitoring component may include one or more sensor components that may assist with monitoring aspects of user health, air quality, operational efficacy, and other metrics that would be understood by those of skill in the art. The monitoring component may additionally include technology capable of detecting presence or absence of potentially hazardous viruses, bacteria, and/or other agents in a user.

The sensors included by the monitoring component may detect one or more environmental conditions in the vicinity of a user of the devices. The environmental conditions may include, without limitation, presence or absence of airborne viruses, bacteria, carcinogens, and pollutants, as well as temperature, precipitation, and humidity. The sensor components may be communicatively and operatively connected to a display component capable of displaying data concerning the environmental conditions.

The monitoring component may operate using a computerized device including a processor capable of reading from memory and operating instructions stored in the memory, as will be appreciated by those of skill in the art. Alternatively, the monitoring component may assist with the analysis and presentation of information performed by an external computerized device associated with operation of the monitoring component, without limitation.

In operation, an air purification apparatus enabled by this disclosure may be used for purifying and/or reducing undesirable materials from air intended to enter a nasal cavity. Those of skill in the art will appreciate methods of use enabled by the above disclosure. Skilled artisans will appreciate additional methods within the scope and spirit of the disclosure for performing the operations provided by the examples herein after having the benefit of this disclosure. Such additional methods are intended to be included by this disclosure.

While various aspects have been described in the above disclosure, the description of this disclosure is intended to illustrate and not limit the scope of the invention. The invention is defined by the scope of the appended claims and not the illustrations and examples provided in the above disclosure. Skilled artisans will appreciate additional aspects of the invention, which may be realized in alternative embodiments, after having the benefit of the above disclosure. Other aspects, advantages, embodiments, and modifications are within the scope of the following claims.

What is claimed is:

1. An air purification apparatus for nasal application comprising:
a housing to substantially enclose an interior space;
air treatment components located within the interior space to at least partially purify air for delivery to a nasal cavity comprising:
a filtration component to remove impurities from the air, and
a fan component to actively pass the air through the filtration component;
a power component located substantially within the interior space to provide electrical power to the air treatment components; and
a case to receive at least part of the housing to be removably stored by the case.

2. The apparatus of claim 1, wherein the power component further comprises:
a battery to reversibly store the electrical power;
power delivery electronics to transmit the electrical power from the battery to the air treatment components; and
battery charging electronics to receive external power from an external power source, condition the external power to a charging power receivable by the battery, and store at least part of the charging power in the battery for use by the air treatment components as the electrical power.

3. The apparatus of claim 1, wherein the housing comprises:
a top housing portion,
a bottom housing portion removably attached to the top housing portion, and
housing inlets located on the bottom housing portion to permit the air to flow into the interior space;
wherein the fan component receives the air from the housing inlets to be passed through the filtration component; and
wherein the air that is passed through the filtration component is moved to the nasal cavity.

4. The apparatus of claim 3, wherein the housing further comprises:
a locking interface comprising:
a locking interface top portion operatively attached to the top housing portion,
a locking interface bottom portion operatively attached to the bottom housing portion,
a locking member provided by the locking interface top portion to receive at least part of a locking lip of the locking interface bottom portion, and
a locking gap provided by the locking interface bottom portion having sufficient width to receive the locking member;
wherein the locking interface is reversibly configured in a locked position by rotating the locking member passed through the locking gap in a first locking direction to receive the locking lip; and
wherein the locking interface is reversibly configured in an unlocked position by rotating the locking member in a second locking direction to release the locking lip.

5. The apparatus of claim 1, wherein the air treatment components further comprise:
a germicidal irradiation component to emit ultraviolet electromagnetic radiation that at least partially sterilizes pathogens from the air prior to entry into the nasal cavity.

6. The apparatus of claim 1, wherein the air treatment components further comprise:
an aroma component comprising an aroma reservoir to reversibly hold an aromatic material having an aroma detectable by an olfactory nerve; and
wherein the aroma is at least partially transferred from the aromatic material into the air to be delivered to the nasal cavity.

7. The apparatus of claim 1, wherein the filtration component comprises a high efficiency particulate absorbing (HEPA) filter.

8. The apparatus of claim 1, further comprising an olfactory substance delivery component capable of administering a deliverable substance.

9. The apparatus of claim 8, wherein the deliverable substance comprises a pharmacological substance.

10. The apparatus of claim 1:
wherein the case comprises:
a case enclosure comprising a case receiving slot to selectively receive the housing,
a case lid being removably installable to the case enclosure to configure the case between a case open state and a case closed state, and
electrical contacts located within the case receiving slot to interface with the power component;
wherein the power component further comprises:
a battery to reversibly store the electrical power received from the case for use by the air treatment components;
wherein the housing is selectively inserted into the case receiving slot or selectively removed from the case receiving slot when the case is configured in the case open state; and
wherein such housing being received by the case receiving slot is substantially enclosed by the case while the case is configured in the case closed state.

11. The apparatus of claim 1, further comprising:
a monitoring component comprising a sensor to detect a condition of the air included by an environmental space adjacent to the sensor; and
wherein the condition is reportable via a communicably connected display device.

12. An air purification apparatus for nasal application comprising:
a housing to substantially enclose an interior space;
a nose plug component operatively attached to the housing to be removably inserted into a nostril such to form an at least partial seal with a nasal cavity;
air treatment components located within the interior space to at least partially purify air for delivery to the nasal cavity comprising:
a germicidal irradiation component to emit ultraviolet electromagnetic radiation that at least partially sterilizes pathogens from the air prior to entry into the nasal cavity;
a power component located substantially within the interior space to provide electrical power to the air treatment components; and
wherein the housing is selectively received by and selectively removed from a case.

13. The apparatus of claim 12, wherein the air treatment components further comprise:
a filtration component to remove impurities from the air; and
a fan component to actively pass the air through the filtration component.

14. The apparatus of claim 12, wherein the housing comprises:
a top housing portion;
a bottom housing portion removably attached to the top housing portion; and
housing inlets located on the bottom housing portion to permit the air to flow into the interior space.

15. The apparatus of claim 12, wherein the power component further comprises:
a battery to reversibly store the electrical power;
power delivery electronics to transmit the electrical power from the battery to the air treatment components; and
battery charging electronics to receive external power from an external power source, condition the external power to a charging power receivable by the battery, and store at least part of the charging power in the battery for use by the air treatment components as the electrical power.

16. The apparatus of claim 12, wherein the air treatment components further comprise:
an aroma component comprising:
an aroma reservoir to reversibly hold an aromatic material having an aroma that is at least partially diffused into the air to be detectable by an olfactory nerve.

17. The apparatus of claim 12, wherein the filtration component comprises a high efficiency particulate absorbing (HEPA) filter and a carbon filter.

18. The apparatus of claim 12:
wherein the case further comprises:
a case enclosure comprising a case receiving slot to selectively receive the housing,
a case lid being removably installable to the case enclosure to configure the case between a case open state and a case closed state, and
electrical contacts to interface with the power component;
wherein the power component further comprises:
a battery to reversibly store the electrical power received from the case for use by the air treatment components;
wherein the housing is selectively inserted into the case receiving slot or selectively removed from the case receiving slot when the case is configured in the case open state; and
wherein such housing being received by the case receiving slot is substantially enclosed by the case while the case is configured in the case closed state.

19. The apparatus of claim 12, further comprising:
an olfactory substance delivery component capable of administering a deliverable substance to the nasal cavity.

20. The apparatus of claim 12, further comprising:
a monitoring component comprising a sensor to detect a condition of the air included by an environmental space adjacent to the housing; and wherein the condition is reportable via a communicably connected display device.

21. An air purification apparatus for nasal application comprising:
a housing to substantially enclose an interior space;
air treatment components located within the interior space to at least partially purify air for delivery to a nasal cavity comprising:
a filtration component to remove impurities from the air, and
an aroma component comprising an aroma reservoir to reversibly hold an aromatic material having an aroma detectable by an olfactory nerve, wherein the aroma is at least partially transferred from the aromatic material into the air to be delivered to the nasal cavity.
